(19) United States
(12) Patent Application Publication
(10) Pub. No.: US 2007/0191795 A1
(43) Pub. Date: Aug. 16, 2007

(54) DISPOSABLE URINATION DEVICE

(76) Inventor: LEO CARMINE DI CROCE, TORONTO (CA)

Correspondence Address: BERESKIN AND PARR, 40 KING STREET WEST, BOX 401, TORONTO, ON, M5H 3Y2, US

(21) Appl. No.: 11/673,124

(22) Filed: Feb. 9, 2007

Related U.S. Application Data
(60) Provisional application No. 60/771,856, filed on Feb. 10, 2006.

(51) Int. Cl. A61F 5/44

(52) U.S. Cl. 604/347

(57) ABSTRACT

A disposable urinary device for collecting urinary fluid from a user and directing it into a stream includes an extendible body, a top section and a bottom end section. The top section is coupled to the top of the extendible body around a first opening and has a sidewall that defines a top opening. The bottom end section is coupled below the extendible body around a second opening and has a bottom opening. The extendible body defines an axial passage between the first and second openings and includes a plurality of flexible bellows type folds. The bellows type folds are sufficiently flexible to allow the device to be moveable between an unpackaged position and an extended position. In the unpackaged position, the bottom end section protrudes below the top section to allow a user to grasp the bottom end section. In the extended position, the bellows type folds are extended to form an arc shaped funnel for collecting the urine stream through the top opening and for directing it into a stream that emerges from the bottom opening.

This application claims the benefit under 35 U.S.C. 119(e) of U.S. Provisional Patent Application No. 60/771,856, filed Feb. 10, 2006, the contents of which are incorporated by reference herein.

### FIELD

The embodiments described herein relates to a urinary device and more particularly to an improved disposable urinary device.

### BACKGROUND

The urine stream for women is not as controlled as the urine stream for men due to various basic physiological differences between them. This is the reason why male style urinals are not suitable for use by women. Rather, sit down toilets are generally used by women to dispose of urine that can present many hygienic issues. Many women wish to avoid contact with the seats of public toilets, in situations where sanitary facilities are not available, for roadside stops and for any other situation where a woman would otherwise have to sit, squat, or come in physical contact with unsanitary facilities in order to urinate.

Various urinary devices to aid females to urinate while standing in an upright position have been disclosed in the prior art. However, many prior art urinary devices are not suitable for placement within compact packaging, an essential requirement for efficient use of shelf space in the retail environment and within coin operated dispenser machines. Also, many prior art urinary devices are cumbersome and inconvenient to transport within a bag or purse. Accordingly are not particularly attractive to prospective retailers or end users.

### SUMMARY

The embodiments described herein provide in one aspect, a disposable urinary device for collecting urinary fluid from a user and directing it into a stream, said disposable urinary device comprising:
(a) an extendible body having a first opening and a second opening, said extendible body defining an axial passage that runs from the first opening to the second opening;
(b) a top section coupled to the extendible body around the first opening, said top section having a sidewall that defines a top opening;
(c) a bottom end section coupled to the extendible body around the second opening, said bottom end section having a bottom opening; and
(d) the extendible body having a plurality of flexible bellows type folds such that the device is moveable between a unpackaged position and an extended position, wherein in the unpackaged position the bottom end section protrudes below the top section to allow a user to grasp the bottom end section, and in the extended position the bellows type folds of the extendible body are extended to form an arc shaped funnel for collecting the urinary fluid through the top opening and directed into a stream that emerges from the bottom opening.

Further aspects and advantages of the embodiments described herein will appear from the following description taken together with the accompanying drawings.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a bottom perspective view of an exemplary embodiment of a disposable urinary device in an unpackaged position;

FIG. 2 is a top perspective view of the disposable urinary device of FIG. 1 in an unpackaged position;

FIG. 3 is a side partial cut away cross-sectional view of the disposable urinary device of FIG. 1 in an unpackaged position;

FIG. 4 is a top perspective cut away cross-sectional view of the disposable urinary device of FIG. 1 in an unpackaged position;

FIG. 5 is a bottom view of the disposable urinary device of FIG. 1 in an unpackaged position;

FIG. 6 is a side perspective view of the disposable urinary device of FIG. 1 in an extended position; and

FIG. 7 is a side partial cross-sectional view of an exemplary embodiment of the disposable urinary device of FIG. 1 in a packaged position.

It will be appreciated that for simplicity and clarity of illustration, elements shown in the figures have not necessary been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

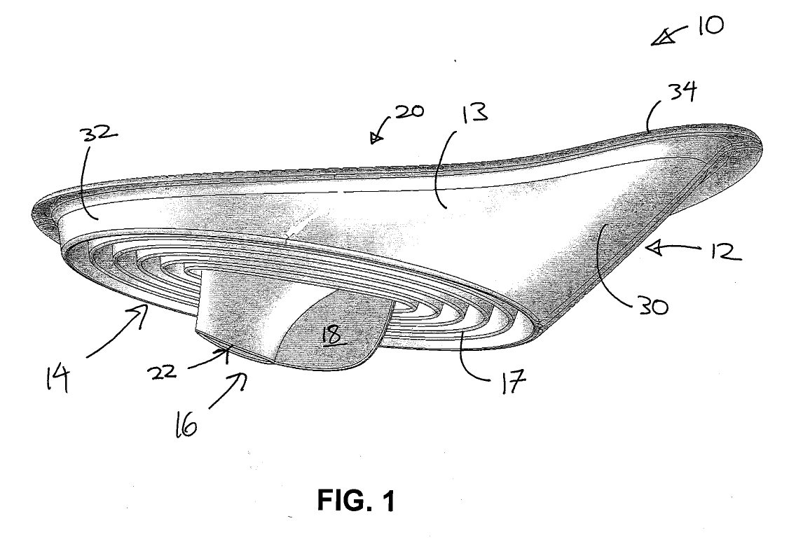

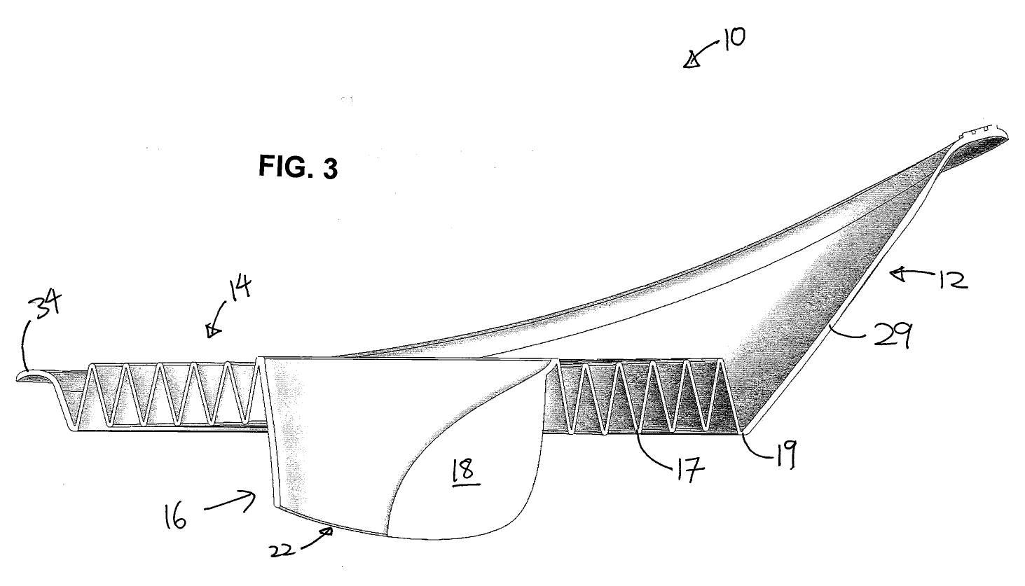

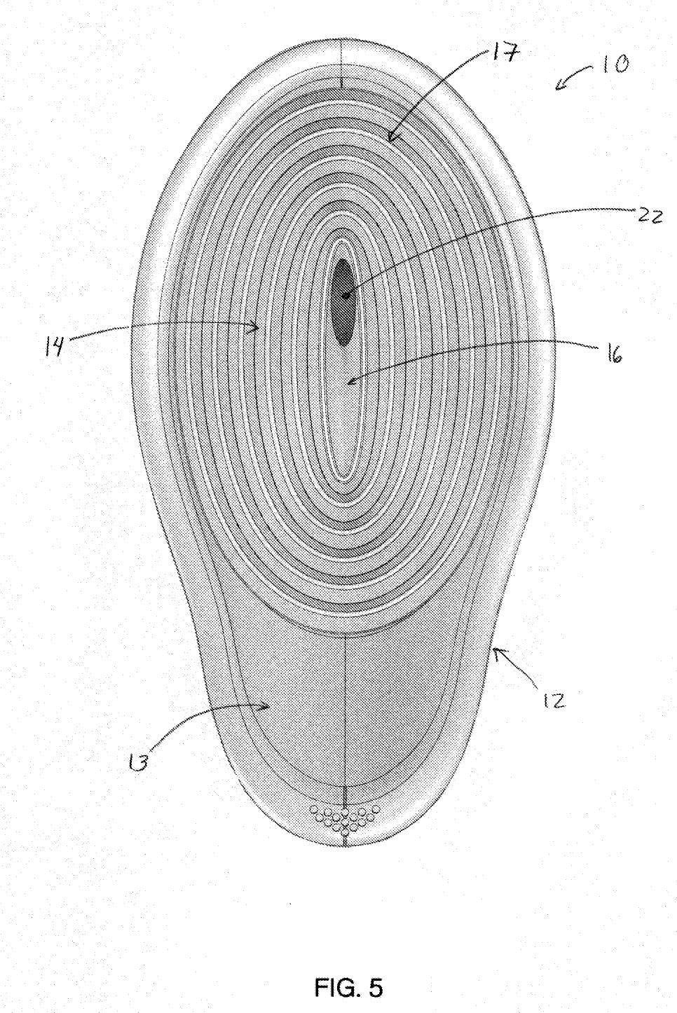

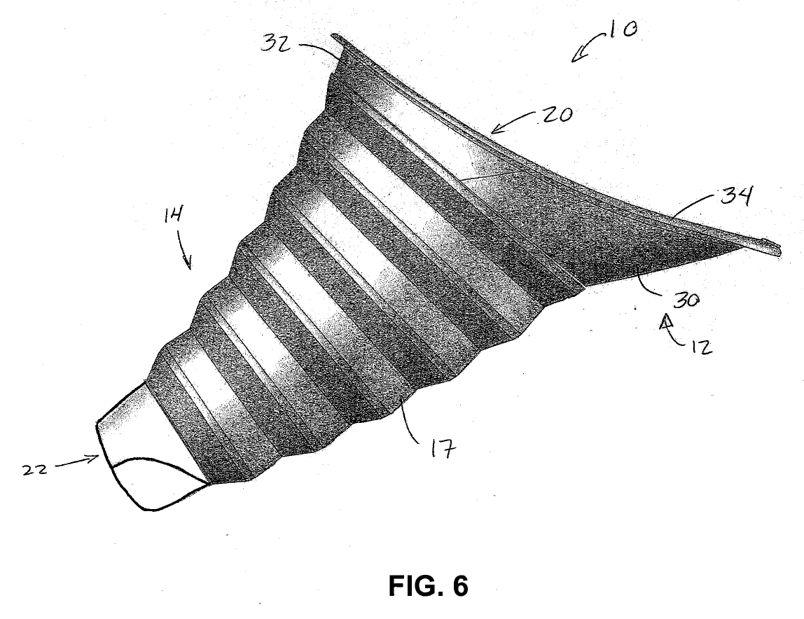

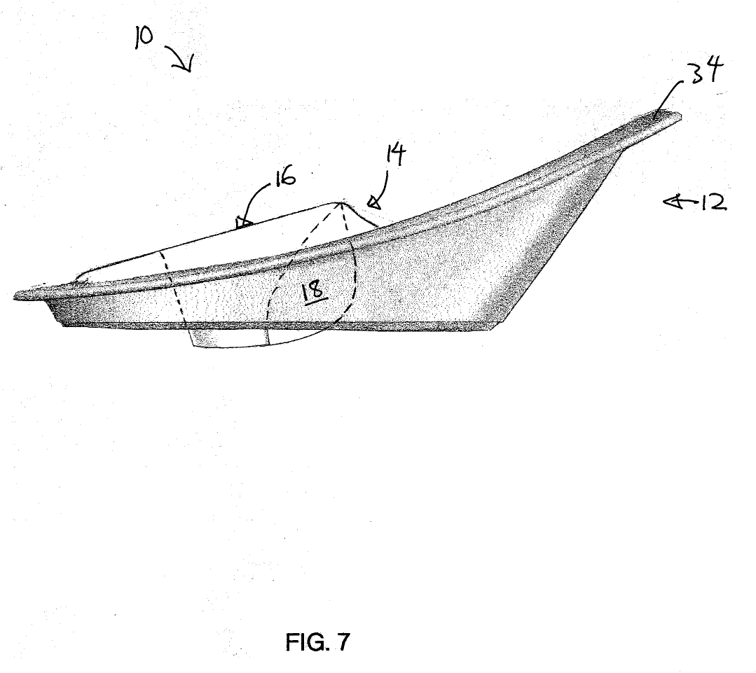

Referring to FIGS. 1 through 5, there is shown an exemplary embodiment of a disposable urinary device 10 in an unpackaged (i.e. manufactured) position. As shown, disposable urinary device 10 comprises a number of specific elements including a top section 12, an extendible body 14, and a bottom end section 16. Disposable urinary device 10 is designed to be manufactured in an unpackaged position (FIGS. 1 to 5), moved into a packaged position (FIG. 7) when packaging is used to wrap disposable urinary device 10 for sale and distribution, and then to be moved into an extended position by the user for use (FIG. 6).

The top section 12 has a sidewall 13 that is generally oval in profile and that includes an elongated bottom section 30 and a foreshortened top section 32. The top section 12 is preferably formed of a moderately rigid material so that sufficient rigidity is maintained for forming and maintaining the opening while at the same time being sufficiently flexible so that it may be manipulated to accommodate various dimensions of the user's body. For example, the top section 12 can be made from a plastic material compound such as propylene copolymer in combination with thermoplastic elastomer.

The top section 12 includes a lip 34 that defines a top opening 20 and a bottom ridge 21 that interfaces with the top of the extendible body 14. The lip 34 is designed to substantially correspond to the contour of the female body where the disposable urinary device is to be placed in use. Specifically, the portion of the lip 34 positioned over the elongated bottom section 30 curves slightly outwards as shown in FIGS. 1 to 4. Furthermore, the outer circumference of the top opening 20 is substantial in size as compared to the average dimensions of the urinary region of the user's body to which the device 10 is designed to be applied. Both of these design features are intended to provide a user with assurance that the disposable urinary device 10 is providing sufficient coverage and that spillage from the top section 12 will be minimized.

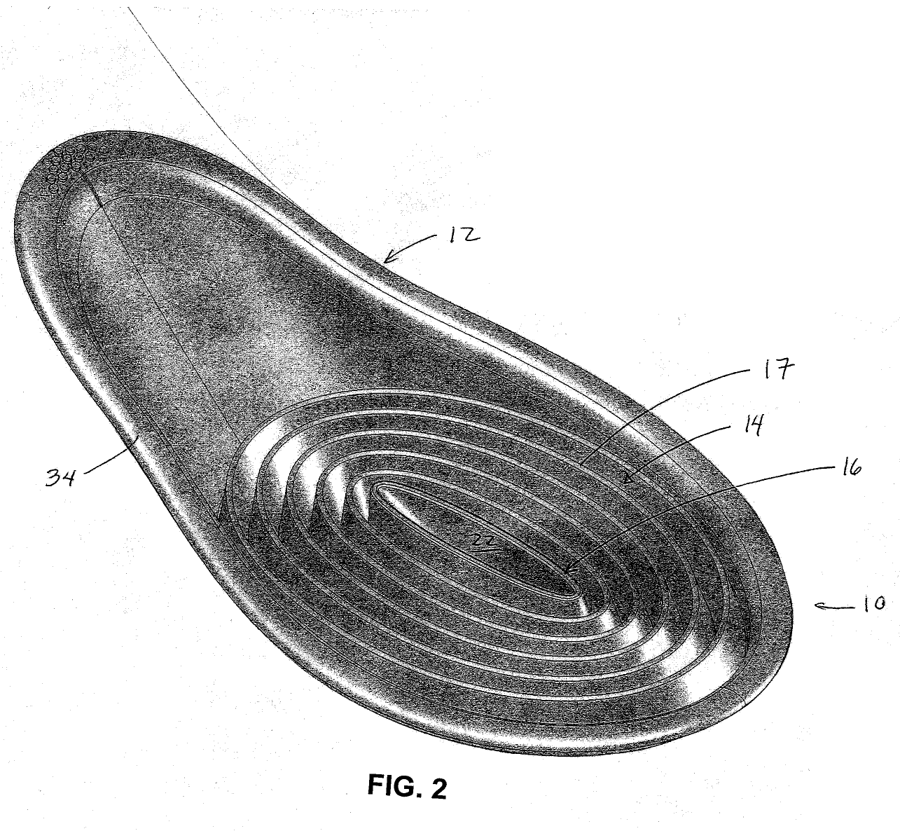

The extendible body 14 consists of a plurality of bellow type folds 17 (FIGS. 1 to 5). As best shown in FIGS. 2 and 6, the bellow type folds 17 decrease in diameter along the longitudinal length of the extendible body 14 towards the bottom end section 16. The plurality of bellow type folds 17 are also designed to allow the extendible body 14 to be extended into an elongated body that tapers downwardly in an arc towards the bottom end section 16 and towards the top of the disposable urinary device 10.

The bellow type folds 17 are manufactured (i.e. molded) in a “compressed” state which can then be expanded to form an arc shaped funnel like profile (FIG. 6) as will be discussed. Since the bellow like folds 17 are molded in a compressed format, no labour is required to flatten out the disposable urinary device 10 before packaging. In addition, the compressed profile provides a compact form that is suitable for distribution, sale and ultimately storage by the user in a bag, purse and the like.

The extendible body 14 is preferably formed of an appreciably flexible material for easy movement between unpackaged position and packaged position and easy manipulation by the user from the unpackaged position to the extended position. For example, the extendible body 14 can be made from a plastic material compound such as propylene copolymer in combination with thermoplastic elastomer. The extendible body 14 has a top ridge 23 (FIG. 1) that is formed along the bottom ridge 21 of the top section 12. The extendible body 14 also has a bottom ridge 24 that is formed along the top portion of the bottom end section 16. When extended, the extendible body 14 defines an axial passage that extends between the top ridge 23 and the bottom ridge 24.

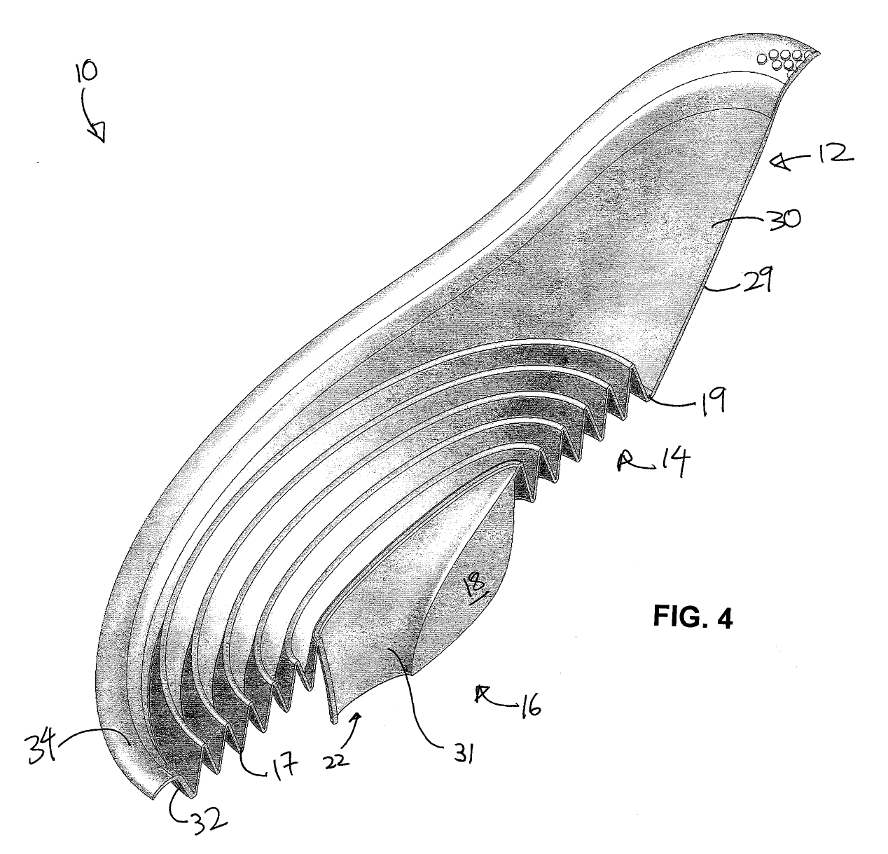

As shown in FIG. 4, the bellow type folds 17 are formed in a uniform manner with generous sized elbows 19 that provide effective extension of the bellow type folds 17. Also, preferably the bellow type folds 17 are manufactured with a thickness that is uniform across all bellow type folds 17 while the thickness of the outer walls 29 of the extendible body 14 is preferably larger. For example, the thickness of the bellow type folds 17 could be 0.02 inches while the thickness of the outer walls 29 of the extendible body 14 could be 0.04 inches.

The flexibility of the flexible bellow type folds 17 allows a user to extend the extendible body 14 away from the device 10 for use by pulling on the extendible tab 18. The bellows like folds 17 are also flexible enough to allow the extendible body 14 to extend upwards into the device 10 such that the device presents a packaging profile that consists substantially of the top section 12 as will be discussed. Accordingly, the use of bellow type folds 17 allows for compact packaging and efficient use of shelf space in the retail environment and within coin operated dispenser machines that are typically located within public toilet facilities. The compact profile also allows convenient transportation of the disposable urinary device 10 within a user's bag or purse and the like.

The bottom end section 16 includes an extension tab 18 that is designed for easy grasping by the user's fingers and which can be easily pulled to stretch out the bellows like folds 17 of the extendible body 14. For example, the extension tab 18 could include gripping elements (e.g. raised segments, or glued on rubber portions) to provide better grip for the user. The bottom end section 16 is preferably formed of a relatively rigid material (e.g. rigid plastic) so that sufficient rigidity is maintained to allow a user to easily and firmly grab the extension tab 18. For example, the bottom end section 16 can be made from a plastic material compound such as propylene copolymer in combination with thermoplastic elastomer.

The bottom end section 16 has an inner surface 31 and bottom opening 22 profile (FIG. 4) that together are adapted to cause the urinary fluid to exit from the disposable urinary device 10 in a spiral-like manner. Specifically, the elliptical profile of the bottom opening 22 where urinary fluid exits the disposable urinary device 10 marshals the “spiral like” motion.

As is conventionally known, surface tension forces generally urge a fluid to move into a round form. Accordingly, in this instance, it is believed that surface tension forces act on fluid exiting the bottom opening 22 urging the elliptical shape of the exiting urinary flow pattern to become rounder in form. As the shape of the urinary flow starts to change (i.e. the major axis of the ellipse gets shorter while the minor axis gets longer) this tendency does not stop when the two axes are the same length and rather it “overshoots”. This results in what used to be the minor axis growing and the major axis shrinking and in this way they reverse roles, effectively creating an ellipse oriented 90 degrees to the original ellipse.

It is believed that this type of motion happens back and forth over the length of the exiting urinary fluid flow until the urinary fluid flow terminates at a surface (i.e. toilet bowl or ground). This back and forth motion generally produces a “spiral” action. Further, it is further believed that under the influence of the well known Coriolis force (i.e. a type of inertial force that affects moving objects in a rotating system, causing them to curve in one direction or another) this alternating motion is further changed into a swirling motion.

This spiral and swirling like characteristic of the urinary fluid flow as it exits disposable urinary device 10 mimics the motion of urine from a male penis and effectively reduces splash back. The bottom end section 16 also acts to somewhat restrict the flow rate of the user's urine stream within the disposable urinary device 10 which also reduces splash back.

It should be understood that while the profile of the bottom opening 22 of the bottom end section 16 has been discussed as being elliptical in shape, any suitably shaped profile could be used. For example, the profile of the bottom opening 22 of the bottom end section 16 could alternatively be a teardrop shape or circular shape.

Also, while it is preferred to manufacture disposal urinary device 10 as one piece from the same material, it should be understood that the top section 12 extendible section 14 and the bottom section 16 could be manufactured from different materials and/or manufactured separately and then assembled (e.g. using conventional adhesion techniques) into a unitary body.

Also, it should be understood that the particular shape and relative sizes of the top section 12, extendible body 14, and bottom end section 16 could be varied from those illustrated in the present figures as long as they present a suitable conduit for directing urinary fluid from the top opening 21 to the bottom opening 22. For example, the depth of top section 12 could be reduced from that shown in FIGS. 1 to 7 in order to allow the disposable urinary device 10 to have a slimmer profile when in the packaged position.

In use, the user extends the disposable urinary device 10 into an extended position (FIG. 6) by grasping the top section 12 in one hand and pulling the extendible tab 18 with the other. Once the disposable urinary device 10 is in the extended position, the user then positions the lip 34 of the top section 12 over their vagina so that the device 10 surrounds the urethral orifice. Also, the user positions the device 10 such that the elongated bottom section 30 is positioned downwards towards the rear of the user and the foreshortened top section 32 is positioned upwards towards the front of the user. During use, the disposable urinary device 10 is designed to temporarily retain whatever volume of urine accumulates. It should be understood that urine will accumulate within the disposable urinary device 10 when the entering flowrate is greater than the exiting flowrate.

Referring now to FIG. 6, when the user grasps the extension tab 18, the user can extend the extendible body 14 into an extended position. Specifically, as mentioned above, the plurality of bellow type folds 17 are designed to allow the extendible body 14 to be extended into an elongated body that tapers downwardly in an arc towards the bottom end section 16 and towards the top of the disposable urinary device 10.

When the extendible body 14 is extended, the resulting arched shaped funnel enables the collection and direction of a user's urine stream through the extendible body 14 so that it is directed into a stream that emerges from the bottom end section 16 that spouts forward and away from the user's feet. The frontward facing direction of the resulting arched shaped funnel also provides an indication to the user as to which way the disposable urinary device 10 should be positioned.

Referring now to FIG. 7 in the packaged position the bottom end section 16 is substantially folded up into the top section 12 when restrained by packaging (not shown). As previously discussed, the extendible body 14 consists of a plurality of bellow type folds 17 such that the extendible body 14 and the bottom end section 16 are together moveable between a packaged position, an unpackaged (i.e. manufactured) position and an extended position.

As discussed above, the bellows like folds 17 are flexible enough to allow a user to extend the extendible body 14 away from the device 10 by pulling on the extendible tab 18. The bellows like folds 17 are also flexible enough to allow the extendible body 14 to extend upwards into the device 10 as shown in FIG. 7. Accordingly, when constrictive packaging is applied to the disposable urinary device 10 after manufacture (FIGS. 1 to 4), the packaging (not shown) will put pressure on the bottom end section 16.

This pressure in combination with the fact that the bellows like folds 17 are flexible enough to allow the extendible body 14 to extend and contract, serves to substantially compress the bottom end section 16 within the top section 12. In this way, the disposable urinary device 10 presents a packaging profile that consists substantially of the top section 12 as will be discussed. This provides a compact and relatively flat profile for the disposable urinary device 10 and a convenient format for distribution and sale.

When the disposable urinary device 10 is unwrapped by the user (i.e. the packaging is removed), the pressure exerted by the packaging on the bottom end section 16 will no longer be present. Accordingly, the bottom end section 16 will return to the position in which it was manufactured and the bottom end section 16 and specifically the extension tab 18 will protrude downwards and away from the top section for easy grasping by the user.

While the above-noted description of the disposable urinary device 10 was considered in relation to application by female users, it should be understood, however, that the utility of the disposable urinary device 10 is not limited to use by females but could also be used by males.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A disposable urinary device for collecting urinary fluid from a user and directing it into a stream, said disposable urinary device comprising:
(a) an extendible body having a first opening and a second opening, said extendible body defining an axial passage that runs from the first opening to the second opening;
(b) a top section coupled to the extendible body around the first opening, said top section having a sidewall that defines a top opening;
(c) a bottom end section coupled to the extendible body around the second opening, said bottom end section having a bottom opening; and
(d) the extendible body having a plurality of flexible bellows type folds such that the device is moveable between a unpackaged position and an extended position, wherein in the unpackaged position the bottom end section protrudes below the top section to allow a user to grasp the bottom end section, and in the extended position the bellows type folds of the extendible body are extended to form an arc shaped funnel for collecting the urinary fluid through the top opening and directed into a stream that emerges from the bottom opening.

2. The disposable urinary device of claim 1, wherein the top section has a lip that runs around the top of the sidewall, wherein the lip substantially corresponds to the contour of the user's body where the disposable urinary device is to be placed for use.

3. The disposable urinary device of claim 1, wherein the top section is generally oval in profile.

4. The disposable urinary device of claim 1, wherein the top section is generally round in profile.

5. The disposable urinary device of claim 1, wherein the sidewall of the top section includes an elongated bottom section and a foreshortened top section.

6. The disposable urinary device of claim 1, wherein the bottom section includes an extension tab that is adapted to be grasped by the user to move the device from the folded position to the extended position.

7. The disposable urinary device of claim 1, wherein the bottom section has an inner surface and wherein the inner surface and the profile of the bottom opening of the bottom end section are adapted to cause the urinary fluid to flow out of the bottom opening in a spiral and swirling like manner.

8. The disposable urinary device of claim 1, wherein the profile of the bottom opening is elliptical wherein the exit profile is elliptical shaped.

9. The disposable urinary device of claim 1, wherein the profile of the bottom opening is elliptical wherein the exit profile is teardrop shaped.

10. The disposable urinary device of claim 1, wherein the diameter of the bellow type folds decrease in diameter along the longitudinal length of the extendible body towards the bottom end section when the device is extended.

11. The disposable urinary device of claim 1, wherein the disposable urinary device is moveable between a packaged position and the unpackaged position wherein in the packaged position the bottom end section is substantially folded up into the top section when restrained by constricting packaging and in the unpackaged position the bottom end section protrudes downwards and away from the top section to allow a user to grasp the bottom end section.
